(19) United States
(12) Patent Application Publication
(10) Pub. No.: US 2018/0289129 A1
(43) Pub. Date: Oct. 11, 2018

(54) MAKEUP APPLICATOR UTILITY CASE

(71) Applicant: Brittany Kosmach, Wickliffe, OH (US)

(72) Inventor: Brittany Kosmach, Wickliffe, OH (US)

(21) Appl. No.: 15/481,285

(22) Filed: Apr. 6, 2017

(51) Int. Cl. A45D 33/26; A61F 13/38; A61F 15/00

(52) U.S. Cl. CPC A45D 33/26; B65D 75/58; A61F 15/001; A61F 13/38

(57) ABSTRACT

A makeup utility case for use by individuals to remove, touchup or reapply makeup, especially around one's eyes, is disclosed. The small utility case is configured to hold a plurality of dual-end applicators, a liquid compartment that maintains a natural makeup removing liquid, and a mirror surface. In usage, the user removes an applicator from the case, applies the makeup removing liquid to one end of the applicator, uses the applicator to remove or touchup her or his makeup, uses the opposite or dry end of the application to dry and further touchup the user's makeup, and then disposes of the applicator. The applicators may be designed and manufactured using biodegradable materials. The liquid containing compartment may be fabricated into one end of the case, or may be located in at least one removable container attached to one surface of the utility case.

### FIELD OF THE INVENTION

The present invention generally relates to the field of makeup devices and cases and related tools, and more specifically relates to a devices, tools, and systems to allow makeup wearers to remove, touchup, or cleanup makeup, especially around the wearer's eyes. In further particularity, an embodiment of the present invention includes a small case comprising a plurality of makeup applicators, a container for a liquid makeup remover, and a mirror to enhance use of the utility case.

### BACKGROUND DESCRIPTION OF THE INVENTION

Wearers of makeup often find a need to touchup their makeup, especially around or near their eyes. Such needs to touchup or cleanup can occur at most any time of the day or evening, and often may occur when the makeup wearer is not at a location where a restroom or mirror is readily available. That is, makeup may be smudged, smeared, or be inadvertently wiped off at inopportune times. Accordingly, having ready access to makeup removing or makeup cleanup tools is an issue or problem that vexes most makeup wearers, and has not to date been adequately addressed.

A key part of the problem is having ready access to a variety of applicator and cleanup tools and components. What is often needed are: a soft and small applicator that is gentle to one's skin for applying a cleaning or makeup removing material; a liquid material to help with removing or touching up makeup; a separate soft and small applicator that can be used to remove or touchup makeup; a mirror that can be used to see the areas of makeup to be cleaned, removed, or touched up; and a case, container, or bag to compactly hold all of these elements in a ready to use fashion. Based upon research, such a device or set of tools has not been designed or commercialized.

Other devices and systems have been designed to address some of these problems and issues, however none appear to adequately resolve all the noted issues. Examples of prior devices and systems include several designed disclosed in U.S. patent applications and granted U.S. patents.

One example device is disclosed in U.S. Pat. No. 5,996,780 for a Cosmetic Apparatus, issued to Gurrera (the “'780 patent” or “Gurrera”). Gurrera teaches a design for a “cotton or foam swab [that] is individually wrapped in a sterilized plastic-lines aluminum foil package having a tear-open tab or strip, and the swab has a lip portion which is saturated with a combination of an anti-aging cream and an eye/face make-up remover.” While Gurrera shows in FIG. 1 a box 11 to contain individually-wrapped applicators, the box 11 by Gurrera does not include any other beneficial features such as a compartment to hold a makeup removing liquid, or a mirror, or other helpful features.

Another example device is described in U.S. Pat. No. 5,947,986 for a Hygienic Applicator, issued to Lewis (the “'986 patent” or “Lewis”). The '986 patent describes and discloses a hygienic applicator having a combination of moist and/or dry swab end sections made of absorbent material. As disclosed in Lewis, the applicator is formed with two ends that are pre-moistened with a desired liquid, such as a medication, and then each end is pre-wrapped to maintain the liquid within the applicator end. Lewis also describes in a different embodiment, a dispenser 50 having a base section 70 that contains a desired liquid. The disclosed dispenser only provides a receptacle to hold the applicators within a base section containing a desired liquid. There are no other disclosures for any other features such as a way to house the applicators in a dry state, or an integral mirror, or a means to replace the liquid containing compartment.

A further device is shown in U.S. patent application Ser. No. 13/169,328 for a Cosmetic Correction Tool, submitted by Sneed (the “'328 application” or “Sneed”) and shows a cosmetic correction tool comprising a stick with a first and second end, which each end having an absorbent swab and a wiping layer covering the absorbent swab. There is no disclosure in Sneed for any type of utility case or a case having a sub-compartment to house a makeup removing material, or having any other features such as a built-in mirror.

Accordingly, while certain designs and products have been created and commercialized to address some of these makeup touchup problems, none appear to provide an acceptable comprehensive solution for makeup touchup issues. What is needed is a compact, easily stored apparatus or case that houses a plurality of makeup applicators along with a natural or hypoallergenic makeup removing solution for easy and clean removal and touchup of a wearer's makeup. Such a device should preferably include a mirror to allow easy viewing of where touchup may be necessary. Moreover, the applicators should preferably be dual-sided, be disposable, and be biodegradable or recyclable. Such an apparatus or set of tools has not been created, disclosed, or used in the prior art.

### SUMMARY OF THE INVENTION

The present invention overcomes the disadvantages of the prior art and fulfills the needs described above by a makeup utility case having a plurality of dual-end applicators along with a container to hold a small amount of makeup removing liquid, and a mirror for use in removing a user's makeup.

One preferred embodiment of the invention is a makeup utility case, comprising a case having at least one liquid holding compartment formed within the case; a plurality of dual-end applicators fitting and stored within the case; a makeup removing liquid maintained within the at least one liquid holding compartments; a mirror surface formed onto a surface of the case; wherein each of the dual-end applicators have at one end a cloth pad used to apply the makeup removing liquid, and have at the other end a cloth pad used to remove the makeup removing liquid and a portion of a wearer's makeup.

Another embodiment of the invention is a makeup utility case comprising a case having at least one liquid holding compartment formed within the case; a plurality of dual-end applicators fitting and stored within the case; a makeup removing liquid maintained within the at least one liquid holding compartments; a mirror surface formed onto a surface of the case; wherein each of the dual-end applicators have at one end a cloth pad used to apply the makeup removing liquid, and have at the other end a cloth pad used to remove the makeup removing liquid and a portion of a wearer's makeup, wherein the dual-end applicator has one cloth pad end to absorb and apply the makeup removing liquid (a wet end), and the other end has a cloth pad to remain dry (a dry end), and further wherein each of the at least one liquid holding compartments have an integrally formed opening that is formed to only accept said wet end of said applicator.

### BRIEF DESCRIPTION OF THE DRAWINGS OF CERTAIN EMBODIMENTS

To easily identify the discussion of any particular element or act, the most significant digit or digits in a reference number refer to the figure number in which that element is first introduced.

FIG. 1 is a perspective view of an embodiment of the makeup utility case in a partially open configuration and single applicator removed from the case.

FIG. 2 is another overhead perspective view of an embodiment of the makeup utility case in a fully open configuration and a single applicator inserted into the liquid container section.

FIG. 3 is a side view of an embodiment of the makeup utility case as depicted in FIG. 2 and in a fully open configuration.

FIG. 4 is a perspective view a second embodiment of the makeup utility case having a liquid container section attached to the case top, and shown in a fully open configuration.

FIG. 5 is another perspective view of the makeup utility case depicted in FIG. 4 shown with an applicator inserted into the liquid container.

FIG. 6 is a perspective view of the makeup utility case shown in FIGS. 4 and 5 in a fully closed configuration.

FIG. 7 is a perspective view another embodiment of the makeup utility case having a liquid container section attached to the case top, and showing use of a lighted mirror.

### DETAILED DESCRIPTION OF CERTAIN EMBODIMENTS

An innovative makeup utility case for use by those who wear makeup, and who wish to have ready access to necessary tools to touchup or cleanup their makeup during the day or night, wherever they may be, is disclosed. The utility case is designed to be compact, yet highly useful because of efficient use of space, ergonomic design of makeup applicators, and inclusion of a natural liquid makeup remover.

In more particularity, and in one primary preferred embodiment, the utility case 10 is structured to have at least two distinct compartments. A first compartment or section is design to removably hold a plurality of applicators 30 or applicator sticks. A second compartment 41 or section is designed to contain a liquid makeup remover 90 that can be readily accessed by one or both ends of one or more of the applicators 30.

In preferred embodiments, the utility case 10 may be partially or fully manufactured using a biodegradable and recyclable material that allows for disposal of the makeup utility case after use, in any recycle container or at any recycle facility. In another embodiment, the utility case 10 may be partially or fully manufactured using resilient thermoplastic material, such as polyvinyl chloride (PVC), acrylonitrile butadiene styrene (ABS), or polycarbonate. Through use of such thermoplastic materials, a blow-molding manufacturing process may be used to rapidly and repeatedly fabricate the utility case 10 to any desired shape.

Alternatively, the utility case 10 may be alternatively manufactured from a lightweight metal such as aluminum. For durability reasons, especially given that the makeup case may often be stored in a purse or pocketbook with a multitude of other items, including keys, cellphones, glasses cases and such, having the case manufactured from a resilient or rigid material could be beneficial to prevent damage to the utility case 10 or any of the contents within the case, including any of the applicators 30 or the second liquid containing compartment 41. The manufacture of the utility case 10 from a metal material also allows for inclusion of various ornamental, decorative, or other luxury design features to be incorporated on the outside of the case 10.

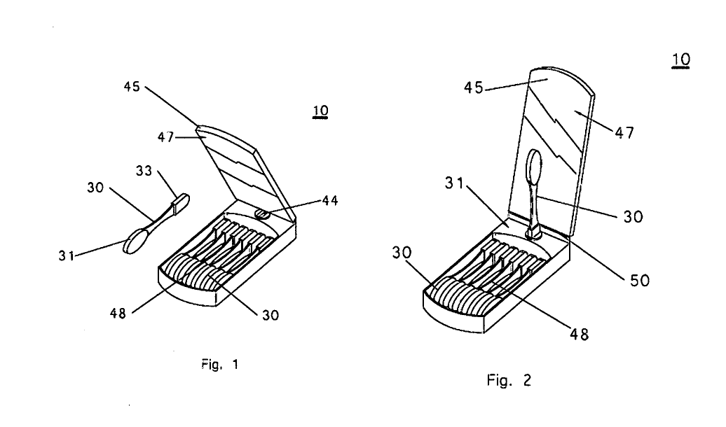

As shown in one embodiment in FIG. 1, the utility case 10 is illustrated in a partially open configuration. The utility case 10 depicted has two primary compartments 48 and 41. The applicator compartment 48 is configured to removably hold a plurality of makeup applicators 30 that are to be used to remove or touch up the makeup. The second compartment, the liquid containing compartment 41 is designed to hold the liquid makeup remover 90. As shown, the utility case 10 has a pivotable top element 45 that can be pivoted into a closed position to cover the applicator compartment 48 and hold the applicators 30 in place, or pivoted into an open position, as shown, to allow access to the inside of the utility case 10.

The liquid containing compartment 41 has an opening 44 through which an applicator 30 may be inserted to immerse one or both ends of the applicator 30 in the makeup removing liquid 90. The opening 44 in the liquid containing compartment may be designed with a self-sealing opening such that the liquid cannot easily spill out if the utility case 10 is tilted or turned over.

In the embodiment shown, the utility case top section 45 may be formed with a closing element 43 such that when the top section 45 is fully closed, the closing element 43 further seals the liquid containing compartment opening 44, and thereby maintains the liquid 90 within the compartment 41. Further, as shown in FIGS. 1 and 2, the utility case top section 45 may be formed with an integral mirror surface 47 to allow the user and makeup wearer to see a reflection of themselves and the area of one's face to which application or removal of any makeup is intended.

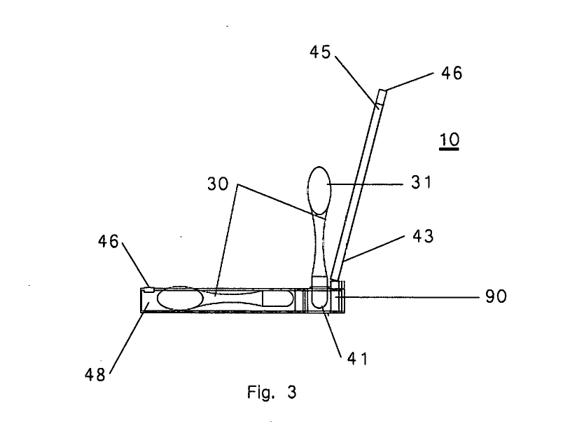

FIG. 3 also shows the incorporation of a physical latch or magnet element 46 in the utility case 10 and top section 45. Use of such a latch or magnet 46 ensures that the makeup utility case does not inadvertently open which could result in applicators 30 falling out of the utility case 10, or the liquid 90 leaking out of the liquid containing compartment 41. FIGS. 2 and 3 also show in one embodiment the use of a hinge 50 that allows the utility case top 45 to readily pivot to an open configuration as shown in FIGS. 1, 2, and 3.

In terms of usage, the makeup wearer may open the utility case 10, remove an unused applicator 30, as shown in FIG. 1, and then dip or insert a cleaning end 33 of the applicator 30 into the liquid containing compartment 41, as shown in FIGS. 2 and 3. With the makeup removing liquid now absorbed into the cleaning end 33 of the applicator 30, the user may easily touchup, cleanup, or remove any makeup the user is wearing. The user may use the mirror 47 to assist in precisely removing or touching up makeup on her or his face. The user may then use the other dry end 31 of the applicator 30 to dry or further clean up the area where the makeup was just removed. After use, the applicator 30 may be readily disposed.

As noted, in a preferred embodiment, the applicator is manufactured from biodegradable and recyclable materials, so easy disposal of used applicators 30 is available. Moreover, because the makeup removing liquid 90 is a natural, hypoallergenic material, ready disposal of the applicator 30 soaked with such material is not an issue.

As illustrated in FIGS. 1 through 6, and highlighted in the side view of FIG. 3, in a preferred embodiment, the applicator 30 may be formed with different shapes for the two ends of the applicator 30. More particularly, there may be a cleaning end 33 and a drying end 31. By using different shapes for the two ends of the applicator 30, the liquid containing compartment opening 44 may be formed such that only the cleaning end 33 fits into the liquid containing compartment opening 44, similar to a key having a shape preventing incorrect access. As such, with different shapes for the applicator 30 end sections, only the cleaning end 33 may be dipped in the liquid containing compartment 41 and have access to and absorb the liquid 90.

Moreover, in different embodiments, the applicator 30 ends may be formed using different types of cloth materials or textiles. Obviously soft materials are preferred to ensure sensitive skin is not scratched or injured with either end of the applicators 30. Accordingly, soft and hydrophilic materials are preferred so that the liquid 90 is readily absorbed into the applicator 30 ends, and any remaining liquid or makeup on the wearer's skin is readily absorbed and thereby removed. One such hydrophilic material would be cotton, which is known to be gentle on one's facial skin.

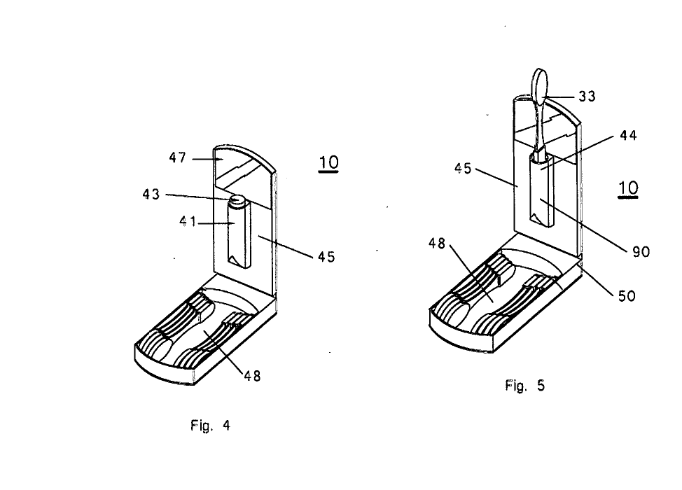

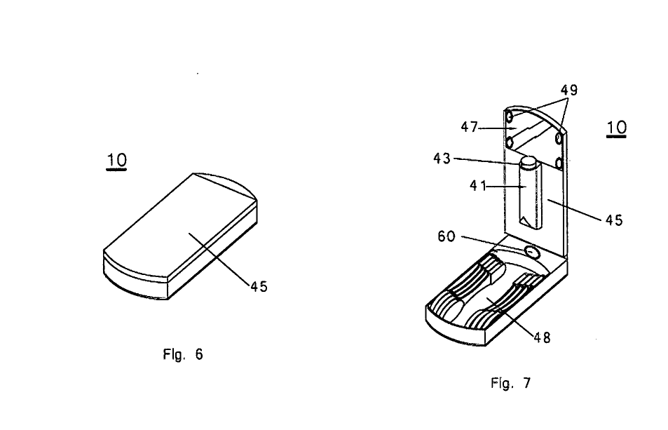

An alternative embodiment of the makeup utility case is shown in FIGS. 4, 5, and 6. In this secondary embodiment, the liquid containing compartment 41 may be formed integrally with the utility case top 45, or may be formed to be removable from the utility case top 45. As illustrated in FIGS. 4 and 5, the liquid containing compartment 41 is formed as part of or attached to the center of the utility case top 45. The utility case 10 body section is accordingly formed with open space to allow the liquid containing compartment to fit within the utility case 10 body section when the utility case top 45 is closed as shown in FIG. 6. The space within the utility case 10 body section is provided by stacking the applicators 30 to either side of the utility case 10 body section as shown in FIGS. 4 and 5.

In this embodiment, the liquid containing compartment 41 has a stopper or top 43 to hold the makeup removing liquid 90 within the compartment 41. For ease of use, the user can open the utility case 10 by rotating the utility case top 45 away from the utility case bottom 53, remove an applicator 30 from the applicator compartment 48, tilting or flipping the top 43 from the liquid containing compartment, dip the cleaning end 33 of the applicator 30 into the liquid containing compartment 41, as shown in FIG. 5. The top 43, should in a preferred embodiment be attached to the top of the liquid containing compartment 41 by a living or flexible hinge.

With the makeup removing liquid now absorbed into the cleaning end 33 of the applicator 30, the makeup wearer may easily touchup, cleanup, or remove any makeup the user is wearing. The wearer may then use the other dry end 31 of the applicator 30 to dry or further clean up the area where the makeup was just removed. As noted above, after use, the applicator 30 may be readily disposed.

By allowing the liquid containing compartment 41 to be removable from the utility case 10, in this embodiment, the utility case 10 may be re-used for multiple applications because the user could remove and refill the liquid containing compartment 41 multiple times. In this embodiment, the liquid containing compartment 41 may be formed from a clear or translucent material to allow the user to see the amount of liquid 90 remaining in the compartment 41.

In a further embodiment, shown in FIG. 7, that provides an additional feature and utility for the user, the mirror 47 may also include light elements 49. Such light elements 49 provide light so that the user can more clearly see the facial areas to be cleaned or touched up even if there is no or limited external or ambient light. For such an embodiment, the light elements 49 could be powered by one or more batteries 60 housed in the utility case 10. For power efficiency, the light elements 49 may be light emitting diodes (“LEDs”). Light elements 49 other than LEDs may alternatively be used, including traditional smaller sized incandescent bulbs.

While several preferred embodiments and features of the inventive makeup utility case 10 have been described and disclosed, in particular with reference to certain figures and drawings showing certain exemplary embodiments that relate to two particular configurations for the case having different locations for the liquid containing section, such cases and the particular designs shown are not to be construed as limiting the scope of the inventive device or inventive products. For example, as described above, the utility case 10 may be configured with the liquid container section embedded or formed in different sections of the utility case, or the overall case may be made in different sizes or shapes for the applicator holding compartment. Further, in other embodiments, two or more separate liquid containing compartments 41 may be included such that different liquids 90 may be contained in the compartments, and thus be available for use by the makeup wearer.

The different shapes for the applicator holding compartment could be formed to hold more or less applicators, or different shaped applicators. Moreover, alternative materials may be used for manufacturing the utility container to provide varied exterior design features and configurations or ornamental features. All such alternate embodiments are believed to be within the scope of the inventive design and the below claims.

It will be recognized by those skilled in the art that other modifications, substitutions, and/or other applications are possible and all such modifications, substitutions and applications are within the true scope and spirit of the present invention. It is likewise understood that the above disclosure and attached claims are intended to cover all such modifications, substitutions, and/or applications.

What I claim as my invention is:

1. A makeup utility case, comprising:
a case having at least one liquid holding compartment formed within said case;
a plurality of dual-end applicators fitting and stored within said case;
a makeup removing liquid maintained within said at least one liquid holding compartment;
a mirror surface formed onto a surface of said case;
wherein each of said dual-end applicators have at one end a cloth pad used to absorb and apply said makeup removing liquid, and have at the other end a cloth pad used to remove said makeup removing liquid and a portion of a wearer's makeup.

2. The makeup utility case of claim 1, wherein said makeup removing liquid is a natural material.

3. The makeup utility case of claim 1, wherein said makeup removing liquid is a natural hypoallergenic material.

4. The makeup utility case of claim 1, wherein said dual-end applicator has one cloth pad end to absorb and apply the makeup removing liquid (a wet end), and the other end has a cloth pad to remain dry (a dry end).

5. The makeup utility case of claim 1, wherein said dual-end applicator cloth pads are formed from a hydrophilic material.

6. The makeup utility case of claim 1, wherein said dual-end applicators have angled end sections for ease of removing makeup around a user's eyes.

7. The makeup utility case of claim 4, wherein each of said at least one liquid holding compartments have an integrally formed opening that is formed to only accept said wet end of said applicator.

8. The makeup utility case of claim 1, wherein said utility case is disposable.

9. The makeup utility case of claim 1, wherein said applicators and said utility case are made of biodegradable materials.

10. The makeup utility case of claim 1, wherein said utility case is manufactured from a metal material.

11. The makeup utility case of claim 1, wherein said utility case is formed with decorative emblems formed into said case exterior surfaces.

12. The makeup utility case of claim 1, wherein said utility case is manufactured from a wood material.

13. The makeup utility case of claim 1, further comprising a top section that is pivotably hinged to said case to enclose said plurality of dual-end applicators when said top section is pivoted to a closed position.

14. The makeup utility case of claim 13, further comprising a magnetic latch component to releasably maintain said top section in a closed position.

15. The makeup utility case of claim 13, further comprising a physical latch component to releasably maintain said top section in a closed position.

16. The makeup utility case of claim 1, wherein said a mirror surface is formed onto an inside top surface of said case.

17. The makeup utility case of claim 1, further comprising at least one light element incorporated along the perimeter of said mirror surface.

18. The makeup utility case of claim 17, wherein said at least one light elements are light emitting diodes.

19. The makeup utility case of claim 17, further comprising at least one battery incorporated into said makeup utility case to power said at least one light element.
